(19) United States
(12) Patent Application Publication
(10) Pub. No.: US 2007/0104781 A1
(43) Pub. Date: May 10, 2007

(54) HYPOCHOLESTEREMIC PREPARATIONS CONTAINING MIXTURES OF PHYTOSTENOL(ESTER)S AND CONJUGATED FATTY ACIDS, AND METHODS OF USING THE SAME

(76) Inventor: BERND FABRY, Korschenbroich (DE)

Correspondence Address: COGNIS CORPORATION;PATENT DEPARTMENT, 300 BROOKSIDE AVENUE, AMBLER, PA, 19002, US

(21) Appl. No.: 11/553,541

(22) Filed: Oct. 27, 2006

Related U.S. Application Data
(63) Continuation of application No. 09/554,387, filed on Jun. 29, 2000, now abandoned.

(30) Foreign Application Priority Data
Nov. 14, 1997 (DE) 197 50 453.1

(51) Int. Cl. A61K 31/56; A61K 9/64; C07J 9/00

(52) U.S. Cl. 424/456; 514/169; 552/540

(57) ABSTRACT

A hypocholesteremic preparation containing at least one component (a) selected from the group consisting of phytostenols and phytostenol esters and at least one component (b) selected from conjugated fatty acids having from about 6 to about 24 carbon atoms and glycerides of conjugated fatty acids having from about 6 to about 24 carbon atoms is disclosed. Methods of reducing serum cholesterol content in a mammal via administration of hypocholesteremic preparations described herein are also disclosed.

### FIELD OF THE INVENTION

The invention relates to the use of synergistic mixtures of phytostenols or phytostenol esters and conjugated fatty acids for producing preparations for decreasing the cholesterol content in the serum of warm-blooded animals.

### PRIOR ART

Hypocholesteremic active agents are understood as meaning preparations which lead to a decrease in the cholesterol content in the serum of warm-blooded animals without an inhibition or lowering of the formation of cholesterol in the blood occurring. Phytostenols, i.e. plant stenols, and their esters with fatty acids have already been proposed for this purpose by Peterson et al. in J. Nutrit. 50, 191 (1953). The patent Specifications U.S. Pat. No. 3,089,939, U.S. Pat. No. 3,203,862 as well as the German Laid-Open Specification DE-A 2035069 (Procter & Gamble) also point in the same direction. The active agents are customarily added to cooking or food oils and then ingested via the food, the amounts employed, however, as a rule being low and customarily below 0.5% by weight in order to prevent the food oils from becoming cloudy or the stenols from being precipitated on addition of water. For use in the foodstuffs area, in cosmetics, pharmaceutical preparations and in the agrarian sector, storage-stable emulsions of the stenol esters in sugar or polyglycerol esters are proposed in European Patent Application EP-A1 0289636 (Ashai). The incorporation of sitostanol esters to decrease the blood cholesterol content in margarine, butter, mayonnaise, salad dressings and the like is proposed in European Patent Specification EP-B1 0594612 (Raision).

The disadvantage, however, is that the phytostenol esters can customarily be added to the food-stuffs only in small amounts, as otherwise there is the danger that they will impair the taste and/or the consistency of the preparations. For a lasting effect on the cholesterol content in the blood, however, the intake of larger amounts of phytostenols or phytostenol esters would be desirable. Furthermore, the rate at which the substances decrease the content of cholesterol in the serum is worthy of improvement. The object of the invention consequently consisted in remedying these deficiencies.

### DESCRIPTION OF THE INVENTION

The invention relates to the use of mixtures of active agents for producing hypocholesteremic preparations with the proviso that
(a) phytostenols and/or phytostenol esters and
(b) fatty acids having 6 to 24 carbon atoms and at least two conjugated double bonds or their glycerides
   are employed.

Surprisingly, it has been found that mixtures of phytostenols or phytostenol esters with conjugated fatty acids or fatty acid glycerides synergistically cause the reduction of the cholesterol content in the blood serum. Encapsulated in gelatin or directly added to foodstuffs, both the mixtures of active agents can be taken orally without problems.

Phytostenols and Phytostenol Esters

Phytostenols (or synonymously phytosterols) are understood as meaning plant steroids which carry a hydroxyl group only on C-3, but otherwise no functional groups. As a rule, the phytostenols have 27 to 30 carbon atoms and a double bond in the 5/6, optionally 7/8, 8/9 or other positions. In addition to these unsaturated species, suitable stenols are also the saturated compounds obtainable by hardening, which are designated stanols and are additionally included by the present invention. Typical examples of suitable phytostenols are, for example, ergostenols, campestenols, stigmastenols, brassica stenols, and preferably sitostenols or sitostanols and in particular β-sitostenols or β-sitostanols. In addition to the phytostenols mentioned, their esters are preferably employed. The acid component of the ester can have its origin in carboxylic acids of the formula (I)
R¹CO—OH (I)
in which R¹CO is an aliphatic, linear or branched acyl radical having 2 to 22 carbon atoms and 0 and/or 1, 2 or 3 double bonds. Typical examples are acetic acid, propionic acid, butyric acid, valeric acid, caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, elaeostearic acid, arachic acid, gadoleic acid, behenic acid and erucic acid, and their technical mixtures, which are obtained, for example, in the pressure cracking of natural fats and oils, in the reduction of aldehydes from Roelen's oxo synthesis or the dimerization of unsaturated fatty acids. Preferred technical fatty acids are those having 12 to 18 carbon atoms such as, for example, coconut, palmitic, palm kernel or tallow fatty acid. The use of esters of β-sitostenol or β-sitostanol with fatty acids having 12 to 18 carbon atoms is particularly preferred. These esters can be produced both by direct esterification of the phytostenols with the fatty acids or else by transesterification with fatty acid lower alkyl esters or triglycerides in the presence of suitable catalysts, such as, for example, sodium ethylate or especially also enzymes [cf. EP-A2 0195311 (Yoshikawa)]. The hypocholesteremic action of phytostenols or phytostenol esters is disclosed, for example, in European Patent Specification EP-B1 0594612 (Raision) and the literature cited therein.
Conjugated Fatty Acids

The term conjugated fatty acids is understood as meaning aliphatic carboxylic acids having 6 to 24, preferably 16 to 18, carbon atoms and at least two double bonds which are conjugated to one another, i.e. are separated by exactly one single bond. Typical examples are the conjugated linoleic acid (CLA) or conjugated fish fatty acids. It is known of conjugated linoleic acid that it has a low hypocholesteremic action; its use in foodstuffs or as a foodstuff supplement, however, is attributed to the fact that it assists the combustion of endogenous fats [cf. EP-B1 0579901, WO 94/16690, WO 96/06605; (WARF)]. Instead of the conjugated fatty acids, the corresponding full or partial esters with glycerol can also be employed for reasons of taste and because of the better fat solubility.

Tocopherols

The mixtures of active agents may contain potentiating agents of the tocopherols type as further constituents. Tocopherols are understood as meaning chroman-6-ols (3,4-dihydro-2-H-1benzopyran-6-ols) substituted in the 2-position by 4,8,12-trimethyl-tridecyl radicals, which obey the formula (II) 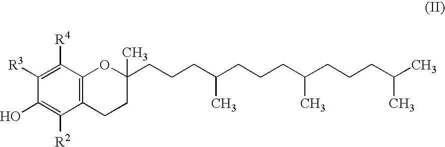
in which R², R³ and R⁴ independently of one another are hydrogen or a methyl group. Tocopherols belong to the bioquinones, i.e. polyprenylated 1,4-benzo- or naphthoquinones whose prenyl chains are saturated to a greater or lesser extent. Typical examples of tocopherols which are possible within the meaning of the invention as component (b) are ubiquinones, boviquinones, K vitamins and/or menaquinones (2-methyl-1,4-naphthoquinones). In the case of the tocopherols, a differentiation is furthermore made between α, β, γ-, δ- and ε-tocopherols, where the latter can still have the original unsaturated prenyl side chain, and α-tocopherolquinone and -hydroquinone, in which the pyran ring system is opened. Preferably, as component (b), α-tocopherol (vitamin E) of the formula (II) is employed, in which R², R³ and R⁴ are methyl groups, or esters of α-tocopherol with carboxylic acids having 2 to 22 carbon atoms, such as, for example, α-tocopherol acetate or α-tocopherol palmitate.
Chitosans

As further constituents, the mixtures of active agents can contain potentiating preparations of the chitosans type. Chitosans are biopolymers and are included in the hydrocolloids group. Considered chemically, they are partially deacetylated chitins of different molecular weights, which contain the following—idealized—monomer unit (III)

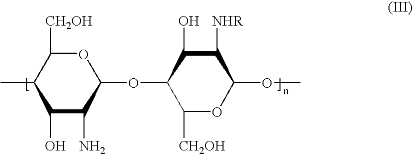

In contrast to most hydrocolloids, which are negatively charged in the biological pH region, chitosans are cationic biopolymers under these conditions. The positively charged chitosans can interact with oppositely charged surfaces and are therefore employed in cosmetic hair- and body-care preparations and pharmaceutical preparations (cf. Ullmann's Encyclopedia of Industrial Chemistry, 5th Ed., Vol. A6, Weinheim, Verlag Chemie, 1986, pp. 231-332). Overviews on this subject have also appeared, for example, by B. Gesslein et al. in HAPPI 27, 57 (1990), O. Skaugrud in Drug Cosm. Ind. 148, 24 (1991) and E. Onsoyen et al. in Seifen-Öle-Fette-Wachse 117, 633 (1991). To produce chitosans, chitin, preferably the shell remains from crustaceans, which are available in large amounts as cheap raw materials, is used as a starting material. In a process which has been described for the first time by Hackmann et al., the chitin is customarily first deproteinated by addition of bases, demineralized by addition of mineral acids and finally deacetylated by addition of strong bases, it being possible for the molecular weights to be distributed over a wide spectrum. Corresponding processes are known, for example, from Makromol. Chem. 177, 3589 (1976) or French Patent Application FR-A 2701266. In a preferred embodiment of the invention, a chitin degradation product, as is described in International Patent Application WO 96/16991 (Henkel), or its degradation product with hydrogen peroxide is employed.

Phytostenol Sulfates

The mixtures of active agents can contain potentiating preparations of the phytostenol sulfates type as further constituents. Phytostenol sulfates are known substances which can be prepared, for example, by sulfation of phytostenols with a complex of sulfur trioxide and pyridine in benzene [cf. J. Am. Chem. Soc. 63, 1259 (1941)]. Typical examples are the sulfates of ergostenols, campestenols, stigmastenols and sitostenols. The phytostenol sulfates can be present as alkali metal and/or alkaline earth metal salts, as ammonium, alkylammonium, alkanolammonium and/or glucammonium salts. As a rule, they are employed in the form of their sodium salts.

(Deoxy)ribonucleic Acids

The mixtures of active agents can finally contain potentiating preparations of the (deoxy)ribonucleic acids type as further constituents. (Deoxy)ribonucleic acids (DNA or RNA) are understood as meaning high molecular weight, threadlike polynucleotides which are derived from 2′-deoxy-β-D-ribonucleosides or D-ribonucleosides, which for their part in turn are synthesized from equivalent amounts of a nucleobase and the pentose 2-deoxy-D-ribofuranose or D-ribofuranose. As nucleobases, the DNA or RNA can contain the purine derivatives adenine and guanine and also the pyrimidines cytosine and thymine or uracil. In the nucleic acids, the nucleobases are linked N-glycosidically with carbon atom 1 of the ribose, adenosines, guanosines, cytidines and thymidines being formed in the individual case. In the acids, a phosphate group links the 5′-hydroxyl group of the nucleosides with the 3′-OH group of the following nucleoside in each case by means of a phosphodiester bridge with formation of single-stranded DNA or RNA. Because of the large ratio of length to diameter, DNA and RNA molecules are prone, even on mechanical stress, for example during extraction, to strand breakage. For this reason, the molecular weight of the nucleic acids can reach 10³ to 10⁹ daltons. Within the meaning of the invention, concentrated DNA and RNA solutions are employed, which are distinguished by a liquid-crystalline behavior. Preferably, deoxy- and ribonucleic acids are employed which are obtained from marine sources, for example by extraction of fish sperm, and which have a molecular weight in the region from 40,000 to 1,000,000 daltons.

Commercial Applicability

The mixtures of active agents of the invention can contain the phytostenols and/or phytostenol esters and the conjugated fatty acids in the weight ratio 99:1 to 1:99, preferably 90:10 to 10:90, in particular 75:25 to 25:75 and particularly preferably 60:40 to 40:60. In a particular embodiment of the invention, the mixtures of active agents are encapsulated in gelatin in a manner known per se, components (a) and (b) in each case being employed in amounts from 0.1 to 50, preferably 1 to 30, in particular 5 to 25 and particularly preferably 10 to 15, % by weight—based on the weight of the gelatin capsules. In addition, it is possible to dissolve or to disperse the mixtures in customary foodstuffs, such as, for example: butter, margarine, dietetic food, deep-frying oils, food oils, mayonnaises, salad dressings, cocoa products, sausage and the like.

### EXAMPLES

### Examples 1 to 5, Comparative Examples C1 to C5

Gelatin capsules (weight about 1.5 g) having a content of 5 or 10% by weight of β-sitostenol or β-sitostenol ester and, if appropriate 5 or 10% by weight of conjugated linoleic acid (CLA) and also 0.5% by weight of radiolabeled cholesterol were prepared. To investigate the hypocholesteremic action, male rats (individual weight about 200 g) were allowed to fast overnight. The following day, a comminuted gelatin capsule was introduced into the experimental animals in each case with some salt-containing water by means of a stomach tube. After 3, 6, 12, 24 and 48 h, blood was taken from the animals and the content of radioactive cholesterol was determined. The results, which represent the mean value of the measurements of 10 experimental animals, are summarized in Table 1. The details on the decrease in the radioactivity are in each case interpreted with respect to a blind group of experimental animals, to which only gelatin capsules having a content of 20% by weight of vitamin E and an appropriate amount of radiolabeled cholesterol had been administered. The mixtures 1 to 5 are according to the invention; the mixtures C1 to C5 serve for comparison.

TABLE 1
Hypocholesteremic action (quantitative data as % by
weight based on gelatin capsule)

| Composition | 1 | 2 | 3 | 4 | 5 | C1 | C2 | C3 | C4 | C5 |
|---|---|---|---|---|---|---|---|---|---|---|
| β-Sitostenol | 5 | — | — | — | — | 10 | — | — | — | — |
| β-Sitostanol | — | 5 | — | — | — | — | 10 | — | — | — |
| Lauric acid β-sitostenol | — | — | 5 | — | — | — | — | 10 | — | — |
| ester | | | | | | | | | | |
| Lauric acid β-sitostanol | — | — | — | 5 | 10 | — | — | — | 10 | — |
| ester | | | | | | | | | | |
| Conjugated linoleic acid | 5 | 5 | 5 | 5 | 5 | — | — | — | — | 10 |

Radioactivity [% rel]

| | | | | | | | | | | |
|---|---|---|---|---|---|---|---|---|---|---|
| after 3 h | 93 | 93 | 93 | 93 | 93 | 93 | 93 | 93 | 93 | 98 |
| after 6 h | 84 | 83 | 83 | 83 | 81 | 87 | 86 | 87 | 86 | 91 |
| after 12 h | 75 | 75 | 75 | 74 | 71 | 79 | 79 | 78 | 78 | 87 |
| after 24 h | 54 | 51 | 47 | 45 | 40 | 62 | 60 | 59 | 69 | 75 |
| after 48 h | 23 | 21 | 22 | 19 | 12 | 35 | 32 | 35 | 32 | 60 |

The examples show the synergistic decrease in the cholesterol content in the blood when using mixtures of the stenols or stenol esters with CLA.

1-10. (canceled)

11. A method of reducing serum cholesterol content in a mammal, said method comprising:
(i) providing a hypocholesteremic preparation comprising at least one component (a) selected from the group consisting of phytostenols and phytostenol esters and at least one component (b) selected from conjugated fatty acids having from about 6 to about 24 carbon atoms and glycerides of conjugated fatty acids having from about 6 to about 24 carbon atoms; and
(ii) administering the hypocholesteremic preparation to a mammal in an amount effective to reduce serum cholesterol content in the mammal.

12. The method according to claim 11, wherein the at least one component (a) is selected from the group consisting of β-sitostenol, β-sitostanol, and esters thereof.

13. The method according to claim 11, wherein the at least one component (a) comprises a carboxylic acid ester of a phytostenol, the carboxylic acid being of the general formula (I):
R¹CO—OH (I)
wherein R¹CO represents an acyl radical having from about 2 to about 22 carbon atoms and up to about 3 carbon-carbon double bonds.

14. The method according to claim 12, wherein the at least one component (a) comprises a carboxylic acid ester of β-sitostenol or β-sitostanol, the carboxylic acid being of the general formula (I):
R¹CO—OH (I)
wherein R¹CO represents an acyl radical having from about 2 to about 22 carbon atoms and up to about 3 carbon-carbon double bonds.

15. The method according to claim 13, wherein the carboxylic acid has from about 12 to about 18 carbon atoms.

16. The method according to claim 14, wherein the carboxylic acid has from about 12 to about 18 carbon atoms.

17. The method according to claim 11, wherein the at least one component (b) comprises conjugated linoleic acid.

18. The method according to claim 11, wherein the hypocholesteremic preparation is encapsulated in gelatin, whereby a gelatin capsule is provided, prior to administering the preparation to the mammal.

19. The method according to claim 18, wherein the at least one component (a) and the at least one component (b) are each independently present in an amount of from about 0.1 to about 50% by weight, based on the total weight of the gelatin capsule.

20. The method according to claim 11, wherein the hypocholesteremic preparation is combined with a foodstuff prior to administering the preparation to the mammal.

21. A hypocholesteremic preparation comprising at least one component (a) selected from the group consisting of phytostenols and phytostenol esters and at least one component (b) selected from conjugated fatty acids having from about 6 to about 24 carbon atoms and glycerides of conjugated fatty acids having from about 6 to about 24 carbon atoms.

22. The hypocholesteremic preparation according to claim 21, wherein the at least one component (a) is selected from the group consisting of β-sitostenol, β-sitostanol, and esters thereof.

23. The hypocholesteremic preparation according to claim 21, wherein the at least one component (a) comprises a carboxylic acid ester of a phytostenol, the carboxylic acid being of the general formula (I):
R¹CO—OH (I)
wherein R¹CO represents an acyl radical having from about 2 to about 22 carbon atoms and up to about 3 carbon-carbon double bonds.

24. The hypocholesteremic preparation according to claim 22, wherein the at least one component (a) comprises a carboxylic acid ester of β-sitostenol or β-sitostanol, the carboxylic acid being of the general formula (I):
R¹CO—OH (I)
wherein R¹CO represents an acyl radical having from about 2 to about 22 carbon atoms and up to about 3 carbon-carbon double bonds.

25. The hypocholesteremic preparation according to claim 23, wherein the carboxylic acid has from about 12 to about 18 carbon atoms.

26. The hypocholesteremic preparation according to claim 24, wherein the carboxylic acid has from about 12 to about 18 carbon atoms.

27. The hypocholesteremic preparation according to claim 21, wherein the at least one component (b) comprises conjugated linoleic acid.

28. The hypocholesteremic preparation according to claim 21, wherein the preparation is encapsulated in gelatin, in order to form a gelatin capsule.

29. The hypocholesteremic preparation according to claim 28, wherein the at least one component (a) and the at least one component (b) are each independently present in an amount of from about 0.1 to about 50% by weight, based on the total weight of the gelatin capsule.

30. The hypocholesteremic preparation according to claim 21, wherein the hypocholesteremic preparation is combined with a foodstuff.

31. The method according to claim 19, wherein the at least one component (a) and the at least one component (b) are each independently present in an amount of from 10 to 15% by weight, based on the total weight of the gelatin capsule.

32. The hypocholesteremic preparation according to claim 29, wherein the at least one component (a) and the at least one component (b) are each independently present in an amount of from 10 to 15% by weight, based on the total weight of the gelatin capsule.
